(19) United States
(12) Patent Application Publication
(10) Pub. No.: US 2010/0167369 A1
(43) Pub. Date: Jul. 1, 2010

(54) Biomass As A Sustainable Energy Source

(76) Inventors: Marco J. Castaldi, New York, NY (US); Kartik Chandran, New York, NY (US)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 12/515,475

(22) PCT Filed: Nov. 20, 2007
(86) PCT No.: PCT/US2007/085204

§ 371 Date: Mar. 2, 2010

Related U.S. Application Data
(60) Provisional application No. 60/860,422, filed on Nov. 21, 2006.

(51) Int. Cl. C12P 5/02; C12M 1/00

(52) U.S. Cl. 435/167; 435/289.1

(57) ABSTRACT

Methods and systems for increasing the generation of methane from a biomass are disclosed. In some embodiments, the method includes the following: decomposing a biomass to produce an gaseous effluent including methane; decomposing a portion of the gaseous effluent in the presence of catalysts to form a decomposed stream including hydrogen, carbon monoxide; converting substantially all of the carbon monoxide in the decomposed stream to carbon dioxide to produce a feed stream including hydrogen and carbon dioxide; and mixing the feed stream with the biomass to facilitate decomposition of the biomass. In some embodiments, the system includes a bioreactor for generating methane from a biomass and additional devices for producing a feed stream including hydrogen and carbon dioxide that is recirculated to the bioreactor to accelerate the production of methane. The additional devices include a catalytic reforming reactor and a shift reactor.

### CROSS REFERENCE TO RELATED APPLICATION(S)

This application claims the benefit of U.S. Provisional Application No. 60/860,422, filed Nov. 21, 2006, which is incorporated by reference as if disclosed herein in its entirety.

### BACKGROUND

The ever-increasing global demand for energy has sparked renewed interest in the study of sustainable alternative energy sources. As a result, the demand for fuels having a lower carbon footprint, e.g., methane (CH₄), hydrogen (H₂), etc., has increased.

Methane in the form of natural gas is commonly used as a heating fuel or an alternative fuel for engines in machinery and motor vehicles. Methane is also used in fuel cells and as a feedstock to produce hydrogen and methanol. The successful use of methane as an alternative to carbon fuels sources can provide significant benefits to the environment and impact world politics by decreasing the dependence of countries on petroleum fuels. Methane burns cleaner than other fuels and produces less carbon dioxide (CO₂) or greenhouse gasses.

Methane is typically obtained by extracting it from natural gas fields, but can also be produced by capturing the biogases generated during the fermentation of organic matter, e.g., gases produced in a bioreactor. However, traditional bioreactors generally require lengthy residence times to produce methane and are often difficult to operate.

### SUMMARY

Methods for accelerating the production of methane from a biomass are disclosed. In some embodiments, the method includes the following: decomposing a biomass to produce an gaseous effluent including methane; decomposing a portion of the gaseous effluent in the presence of catalysts to form a decomposed stream including hydrogen, carbon dioxide, and carbon monoxide; converting substantially all of the carbon monoxide in the decomposed stream to carbon dioxide to produce a feed stream including hydrogen and carbon dioxide; and mixing the feed stream with the biomass to facilitate decomposition of the biomass.

Methods for accelerating the generation of a consumable energy from a biomass are disclosed. In some embodiments, the method includes the following: decomposing a biomass to produce an gaseous effluent including methane; decomposing a portion of the gaseous effluent in the presence of catalysts to form a decomposed stream including hydrogen, carbon dioxide, and carbon monoxide; converting substantially all of the carbon monoxide in the decomposed stream to carbon dioxide to produce a feed stream including hydrogen and carbon dioxide; mixing the feed stream with the biomass to facilitate decomposition of the biomass; feeding a portion of the gaseous effluent to a power plant; and generating a consumable energy with a portion of the gaseous effluent.

Systems for accelerating the generation of methane from a biomass are disclosed. In some embodiments, the system includes the following: a bioreactor for decomposing a biomass to produce a gaseous effluent including methane; a catalytic reforming reactor for decomposing a portion of the gaseous effluent in the presence of catalysts to form a decomposed stream including hydrogen, carbon dioxide, and carbon monoxide; a shift reactor for converting substantially all of the carbon monoxide in the decomposed stream to carbon dioxide to produce a feed stream including hydrogen and carbon dioxide; and a conduit from the shift reactor to the bioreactor for directing the feed stream to the bioreactor to facilitate decomposition of the biomass.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a diagram of a system according to some embodiments of the disclosed subject matter; and

FIG. 2 is a diagram of a method according to some embodiments of the disclosed subject matter.

### DETAILED DESCRIPTION

Generally, the disclosed subject matter relates to systems and methods for accelerating the generation of methane from a biomass. The biomass is decomposed and the biogases containing methane are collected. A first portion of the biogases collected is used as a fuel source to generate energy. A second portion of the biogases collected is further processed to produce hydrogen and to remove carbon monoxide. The second portion is then mixed with the biomass to help facilitate the generation of methane.

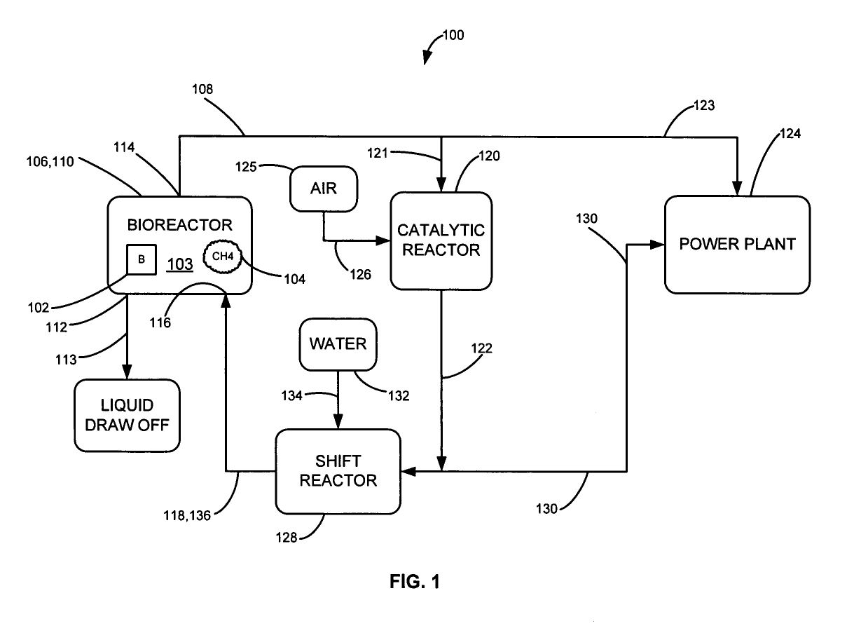

Referring now to FIG. 1, one embodiment of the disclosed subject matter is a system 100 for accelerating the generation of methane 102 from a biomass 104, e.g., a sanitary wastewater, a municipal solid waste (MSW), etc.

In some embodiments, system 100 includes a bioreactor 106 for decomposing biomass 104 to produce a gaseous effluent 108, e.g., a biogas 108 that includes methane 102. Bioreactor 106 is generally, but not always, defined in an enclosed vessel 110 that is configured for holding biomass 104. Vessel 110 includes an outlet 112 for removing liquid 113 from biomass 104 while it is decomposing and an outlet 114 for removing biogas 108. Vessel 110 also includes an inlet 116 for adding a feed stream 118 to bioreactor 106.

In some embodiments, system 100 includes a catalytic reforming reactor 120 for decomposing a portion 121 of gaseous effluent 108 in the presence of catalysts (not shown) to form a decomposed stream 122, which includes hydrogen, carbon dioxide, and carbon monoxide. Portion 121 from bioreactor 106 includes about 10% by weight of gaseous effluent 108. As discussed further below, a remaining portion 123, which is about 90% of gaseous effluent 108, can be sent to a mechanism 124 for generating a consumable energy, e.g., a power generation plant.

The hydrogen generated in catalytic reforming reactor 120 is fed continuously to bioreactor 106. Hydrogen is used by the bacteria in bioreactor 106 as an electron donor for methanogenesis. In most cases, the hydrogen is the limiting reactant. Therefore, feeding hydrogen to bioreactor 106 can help to accelerate the decomposition of biomass 104 and generate a higher flow rate of methane and carbon dioxide.

Catalytic reforming reactor 120 can include a rhodium or nickel catalyst in either a packed-bed or monolith form and at a temperature of between about 550 and 650 degrees Celsius. Nickel catalysts have been found to cost less than a rhodium catalyst over its lifetime of effective use. However, rhodium catalysts have been found to decompose methane at a faster rate and have a lower fouling rate than nickel catalysts. Monolith reactors have been found to have a lower pressure drop than packed bed reactors.

An air source 125 such as, but not limited to, an air compressor or similar is used to provide an air stream 126 required for the operation of catalytic reforming reactor 120. Air stream 126 provides substantially all of the oxygen for a partial oxidation reaction, which will produce the desired hydrogen. In some embodiments, in order to increase the concentration of hydrogen in decomposed stream 122, which is produced by catalytic reforming reactor 120, operating parameters of the catalytic reforming reactor are adjusted, e.g., either an additional amount of the decomposed stream is added or an additional amount of air stream 126 is added, so that during operation it has an equivalence ratio (Ø) of 3.0. The equivalence ratio is defined as:

$$∅ = \frac{{( F / A )}_{actual}}{{( F / A )}_{stoichiometric}} \qquad [ 1 ]$$

where F/A is equal to the fuel (methane) to air (oxygen) ratio.

The effluent of catalytic reforming reactor 120, i.e., decomposed stream 122 generally, but not always, contains a significant amount of carbon monoxide, which is toxic to the bacteria within bioreactor 106. In order to avoid feeding the carbon monoxide to bioreactor 106, system 100 can include a shift reactor 128 positioned after catalytic reforming reactor 120 and before the bioreactor to convert, or shift, the carbon monoxide in decomposed stream 122 to carbon dioxide according to the following:

CO(g)+H₂O [symbol] CO₂(g)+H₂(g) [2].

A portion 130 of decomposed stream 122, which is typically, but not always, rich in hydrogen, can also be sent to mechanism 124.

Shift reactor 128 is used to convert substantially all of the carbon monoxide in decomposed stream 122 to carbon dioxide to produce feed stream 118 including hydrogen and carbon dioxide. The benefits of shifting the carbon monoxide to carbon dioxide are twofold. First, it prevents or substantially reduces the amount of poisonous carbon monoxide in feed stream 118, which is fed to bioreactor 106. Second, it provides bacteria in bioreactor 106 with the species consumed in methane production, i.e., hydrogen and carbon dioxide. A water source 132 is utilized to provide water 134 required for the operation of shift reactor 128. System 100 includes a conduit 136 from shift reactor 128 to bioreactor 106 for directing feed stream 118 to the bioreactor to facilitate decomposition of biomass 104.

Still referring to FIG. 1, system 100 can include or be connected with a mechanism 124 for generating a consumable energy such as, but not limited to, electricity. Portion 123, e.g., about 70 to 90% by weight of biogas 108, can be used as a fuel source to mechanism 124. In some embodiments, mechanism 124 is a methane-powered generator. In some embodiments, mechanism 124 is a fuel cell. As mentioned above, a portion 130 of decomposed stream 122, which is typically, but not always, rich in hydrogen, can also be sent to mechanism 124.

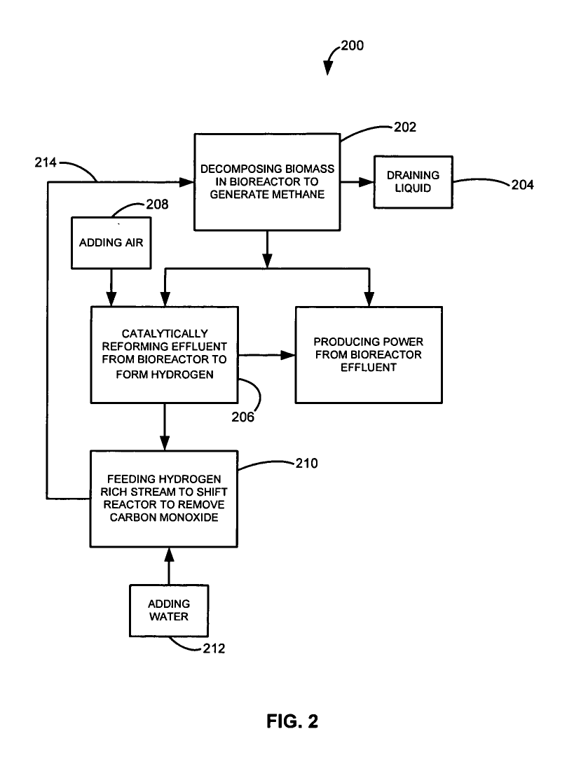

Referring now to FIG. 2, another aspect of the disclosed subject matter is a method 200 of accelerating the production of methane from a biomass such as, but not limited to, a sanitary wastewater, a MSW, etc. At 202, biomass is decomposed to produce a gaseous effluent including methane. At 204, liquid is removed or drained from the biomass while it is decomposing.

In some embodiments, at 202, the biomass is initially decomposed in a traditional batch bioreactor having a mesophilic temperature range of about 30 to 38 degrees Celsius and a pH of about 6.5 to 7.5 to maintain the proper alkalinity. Because a high rate digestion is assumed, in some embodiments, the bioreactor is operated with a residence time of about 10 days. Depending on the actual rate of digestion, as measured, the residence time can be less or greater than 10 days. Approximately two-thirds of the total volume of the bioreactor vessel is charged with an initial amount of MSW. The MSW is simplified to a 50% by weight glucose suspension in water.

At 206, a portion, e.g., about 5% to 30% by weight in some embodiments and about 10% in some embodiments, of the gaseous effluent is decomposed in the presence of catalysts to form a decomposed stream including hydrogen, carbon dioxide, and carbon monoxide. Substantially simultaneous to 206, at 208, air is mixed with the gaseous effluent while it is decomposing in the presence of catalysts.

At 210, substantially all of the carbon monoxide in the decomposed stream is converted to carbon dioxide to produce a feed stream including hydrogen and carbon dioxide. Substantially simultaneous to 210, at 212, water is mixed with the decomposed stream to facilitate production of the feed stream.

At 214, the feed stream is mixed with the biomass to facilitate decomposition of the biomass. After completion of 214, the bioreactor typically, but not always, operates as a semi-batch reactor because the waste that is decomposed by the bacteria is charged in as necessary, which is dictated by the residence time, while the feed stream of hydrogen and carbon dioxide produced at 206 and 210 is fed continuously.

In operation, the initial charge of MSW is allowed to start decomposing at 202 before the external hydrogen and carbon dioxide feed stream is fed into the bioreactor at 214 and for a duration that is sufficient enough to allow substantially all of the fermentative and most of the acetogenic reactions to occur. As this decomposition approaches the end of the acetogenic stage and the beginning of the methanogenic stage, at 214, the continuous feed stream of hydrogen and carbon dioxide is introduced.

As illustrated at 216, in some embodiments, method 200 includes generating a consumable energy with a portion of the gaseous effluent or biogas. For example, a portion of the biogas generated can be used as a fuel source to a methane-powered generator or with a methane fuel cell.

Systems and methods according to the disclosed subject matter provide a sustainable alternative energy source and can be easily integrated into existing wastewater treatment plants. The power generated can be used to operate other conventional equipment within the existing wastewater treatment plant.

Some advantages of systems or methods according to the disclosed subject matter are that it is easily integrated into existing systems and it reduces the treatment costs to the facility while also saving energy. Systems or methods according to the disclosed subject matter can even be used as a stand alone technique in niche applications.

There are numerous benefits to introducing an external feed stream, i.e., feed stream 118 above, into a bioreactor. First, hydrogen and carbon dioxide provide an immediate electron and carbon source for the bacteria. Second, the feed stream increases the contact area between the bacteria and the available food sources. Third, since the external feed stream is at an elevated temperature, it enhances the digestion rate within the bioreactor.

Table 1 includes model results, which show how the external feed stream of hydrogen and carbon dioxide, i.e., feed stream 118 in system 100 above, affects the power generated as compared to a traditional bioreactor system that does not include a feed stream of hydrogen and carbon dioxide.

TABLE 1

| | | Bioreactor Including |
|---|---|---|
| | Traditional | External Feed Stream |
| | Bioreactor | (single pass) |
| Methane produced, lbmol/min | 7.65 | 8.16 |
| Methane sacrificed, lbmol/min | — | 0.765 |
| Methane sent to power plant, | 7.65 | 7.40 |
| lbmol/min | | |
| Biogas Ratio, methane/carbon | 0.89/1 | 0.72/1 |
| dioxide | | |

As shown in Table 1, a bioreactor system having a feed stream of hydrogen and carbon dioxide generated 8.16 lbmol/min of methane as compared to a traditional bioreactor that did not include a feed stream of hydrogen and carbon dioxide, which generated 7.65 lbmol/min of methane. A bioreactor system having a feed stream of hydrogen and carbon dioxide accelerates the decomposition of the biomass by producing more methane.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A method of accelerating the production of methane from a biomass, the method comprising:
decomposing a biomass to produce an gaseous effluent including methane;
decomposing a portion of the gaseous effluent in the presence of catalysts to form a decomposed stream including hydrogen, carbon dioxide, and carbon monoxide;
converting substantially all of the carbon monoxide in the decomposed stream to carbon dioxide to produce a feed stream including hydrogen and carbon dioxide; and
mixing the feed stream with the biomass to facilitate decomposition of the biomass.

2. The method according to claim 1, wherein the biomass is a sanitary wastewater.

3. The method according to claim 1, further comprising removing liquid from the biomass while it is decomposing.

4. The method according to claim 1, further comprising mixing air with the gaseous effluent while it is decomposing in the presence of catalysts.

5. The method according to claim 4, wherein the decomposed stream has an equivalence ratio of 3.0.

6. The method according to claim 1, further comprising mixing water with the decomposed stream to facilitate production of the feed.

7. The method according to claim 1, further comprising generating a consumable energy with a portion of the gaseous effluent.

8. The method according to claim 1, further comprising decomposing the biomass for about 8 to 12 hours.

9. A method of accelerating the generation of a consumable energy from a biomass, the method comprising:
decomposing a biomass to produce an gaseous effluent including methane;
decomposing a portion of the gaseous effluent in the presence of catalysts to form a decomposed stream including hydrogen, carbon dioxide, and carbon monoxide;
converting substantially all of the carbon monoxide in the decomposed stream to carbon dioxide to produce a feed stream including hydrogen and carbon dioxide;
mixing the feed stream with the biomass to facilitate decomposition of the biomass;
feeding a portion of the gaseous effluent to a power plant; and
generating a consumable energy with a portion of the gaseous effluent.

10. The method according to claim 9, further comprising removing liquid from the biomass while it is decomposing.

11. The method according to claim 9, further comprising mixing air with the gaseous effluent while it is decomposing in the presence of catalysts.

12. The method according to claim 9, further comprising mixing water with the decomposed stream to facilitate production of the feed.

13. A system for accelerating the generation of methane from a biomass, the system comprising:
a bioreactor for decomposing a biomass to produce a gaseous effluent including methane;
a catalytic reforming reactor for decomposing a portion of the gaseous effluent in the presence of catalysts to form a decomposed stream including hydrogen, carbon dioxide, and carbon monoxide;
a shift reactor for converting substantially all of the carbon monoxide in the decomposed stream to carbon dioxide to produce a feed stream including hydrogen and carbon dioxide; and
a conduit from the shift reactor to the bioreactor for directing the feed stream to the bioreactor to facilitate decomposition of the biomass.

14. The system according to claim 13, wherein the biomass is a sanitary wastewater.

15. The system according to claim 13, further comprising a means for generating a consumable energy.

16. The system according to claim 15, wherein the means for generating a consumable energy includes a methane-powered generator.

17. The system according to claim 15, wherein the means for generating a consumable energy includes a fuel cell.

18. The system according to claim 15, further comprising a water source for the shift reactor.

19. The system according to claim 15, further comprising an air source for the catalytic reforming reactor.

20. The system according to claim 17, further comprising a conduit from the catalytic reforming reactor to the fuel cell for providing hydrogen to the fuel cell.
